SEQUENCE LISTING (2 sequences)

SEQ ID NO: 1 - DNA, 1199 bp, Populus balsamifera subsp. trichocarpa
  CDS (51)..(977)
gcacgaggttaaacttccttggtttctttcatcaagaaaaattatctacaatggctgataaaagcaaaatcttgatcattggaggtactggttacataggaaaattcatcgtggaggcaagcgccaaggccggtcaccccactttcgccttggttagagagagtacagtctctgatcctgtcaaacgagaacttgtcgagaaattcaagaacttaggcgtcactttgatccatggagatgtcgacggccatgacaatttggtgaaggcaatcaagcgggtggatgtggtgatatcagcgattgggagcatgcaaatagcagatcaaaccaagatcattgccgccattaaagaagctggcaatgtcaagagattcttcccttcagaatttggaatggatgtggatcatgtcaatgctgttgagcctgcaaaaactgcatttgcaatgaaggctcagattcgacgtgccatcgaggctgcagggattccctacacttatgtgccttccaacttctttgctgcatattatctccccacattggcacagtttggacttactgctcctccgagagacaagatcaccatcctcggagatggcaatgccaagttggttttcaataaggaagatgacattggaacctacaccatcaaagctgtggatgatgcaagaaccttgaacaagactgtcctaatcaagcctcctaaaaacacctactcattcaatgagcttattgatctatgggagaaaaagattggcaaaaccctcgaaaaaacctttgttcctgaagagaaacttctgaaggacatccaagagtctccgattccgattaatattgttctgtcaatcaaccactcagccctcgttaatggtgacatgaccaactttgagattgacccatcatggggccttgaggcctctgagctatatccagatgtcaaatataccaccgtggaagagtaccttgatcagtttgtctgaggcactggcatctcctgctctccagttattaatgaaacaaacagccgaatagttggaaatttgggtgtttcttatagacgagtgtttgtccaagtcaaggaggtctctttccttataaaccttgtgaaatgatgttctgctctagttaattgccatggtttgtatctattgctggaagagatgttctgggtgagaataatgtaattgagttatgatgaataa

SEQ ID NO: 2 - protein, 308 aa, Populus balsamifera subsp. trichocarpa
MADKSKILIIGGTGYIGKFIVEASAKAGHPTFALVRESTVSDPVKRELVEKFKNLGVTLIHGDVDGHDNLVKAIKRVDVVISAIGSMQIADQTKIIAAIKEAGNVKRFFPSEFGMDVDHVNAVEPAKTAFAMKAQIRRAIEAAGIPYTYVPSNFFAAYYLPTLAQFGLTAPPRDKITILGDGNAKLVFNKEDDIGTYTIKAVDDARTLNKTVLIKPPKNTYSFNELIDLWEKKIGKTLEKTFVPEEKLLKDIQESPIPINIVLSINHSALVNGDMTNFEIDPSWGLEASELYPDVKYTTVEEYLDQFV

(19) United States
(12) Patent Application Publication
(10) Pub. No.: US 2006/0015967 A1
(43) Pub. Date: Jan. 19, 2006

(54) Role in lignification and growth for plant phenylcoumaran benzylic ether reductase

(76) Inventors: Wout Boerjan, Kalken (BE); Andrea Polle, Duderstadt (DE); Kristine Vander Mijnsbrugge, Merelbeke (DE)

Correspondence Address: NIXON & VANDERHYE, PC, 901 NORTH GLEBE ROAD, 11TH FLOOR, ARLINGTON, VA, 22203, US

(21) Appl. No.: 10/531,479

(22) PCT Filed: Oct. 17, 2003
(86) PCT No.: PCT/EP03/50731

§ 371 Date: Apr. 15, 2005

(30) Foreign Application Priority Data
Oct. 18, 2002 (EP) 02079407.9

(51) Int. Cl. A01H 1/00; A01H 9/00

(52) U.S. Cl. 800/287; 800/295

(57) ABSTRACT

The present invention relates to the role of plant phenylcoumaran benzylic ether reductase (PCBER) (1) in lignification and growth of plants. More particular, the invention relates to plants in which PCBER has been down-regulated, resulting in a lower lignin content, higher soluble phenolics, a higher resistance to plant pathogens and a higher biomass production of the plant. These characteristics are maintained under elevated CO₂ concentrations.

The present invention relates to the role of plant phenylcoumaran benzylic ether reductase (PCBER) in lignification and growth of plants. More particular, the invention relates to plants in which PCBER has been down-regulated, resulting in a lower lignin content, higher soluble phenolics, a higher resistance to plant pathogens and a higher biomass production of the plant. These characteristics are maintained under elevated CO₂ concentrations.

Lignans represent a diverse array of secondary metabolites widely distributed throughout the plant kingdom. They are typically found as dimers of C₆-C₃ phenylpropanoids. Many naturally occurring lignans are 8-8′ linked phenylpropanoid dimers, whereas 8-5′ linked lignans, the phenylcoumarans and sometimes referred to as neolignans, are less common (Ayres and Loike, 1990; Wards, 1997). Despite their ubiquitous presence, the biological significance of lignans in plants is still unclear. Strong evidence argues for an important role in plant defense functions, which is concordant with the view that many secondary metabolic pathways have evolved as deterrents to potential predators or pathogens (Osbourn, 1999). Many reports deal with antimicrobial, antifungal and antifeedant properties of lignans (Hillis, 1987; Davin et al., 1992). Furthermore, biological activities of various lignans in human medicine have been documented: they show antimitotic, antitumor, estrogen-like and antioxidant activities (Ayres and Loike, 1990; Higuchi, 1997; Adlercreutz and Mazur, 1997). A primary biochemical function for lignan glucosides has been demonstrated. Dehydrodiconiferyl alcohol glucosides play a role in the regulation of plant growth through cytokinin-like properties influencing cell division and cell expansion (Binns et al., 1987; Orr and Lynn, 1992; Gaspar et al., 1996; Tamagnone et al., 1998).

In terms of their chemical structure, lignans are closely related to lignin, with which they share common phenylpropanoid precursors (Ayres and Loike, 1990). Both, lignan formation and the first step in monolignol polymerization are considered to arise via bimolecular phenoxy radical coupling. However, lignans differ from the heteropolymerous lignins, as the first are mostly found optically active, whereas the latter do not show any measurable optical activity (Higuchi, 1997).

Some enzymes have been shown to be involved specifically in lignan synthesis, including phenylcoumaran benzylic ether reductase (PCBER). A poplar PCBER has been shown to catalyze the non-enantiospecific NADPH-dependent reduction of the benzylic ether functionality in the 8-5′-linked lignans dehydrodiconiferyl alcohol and dihydrodehydrodiconiferylalcohol (Gang et al., 1999). Remarkably, PCBER is one of the most abundant proteins in xylem (Vander Mijnsbrugge et al., 2000b) and its EST is highly abundant in xylem EST libraries as well (Sterky et al., 1998). PCBER has been detected in all cell types of differentiating xylem and in differentiating phloem fibers of both poplar (Vander Mijnsbrugge et al., 2000a, b) and pine (Kwon et al. 2001). Because of the close association with lignifying cells, it has been hypothesized that PCBER may be involved in the infusion of lignans in the secondary cell wall (Vander Mijnsbrugge et al., 2000b). On the other hand, the well-known antioxidant properties of lignans may point towards a protective role of PCBER during lignification, a process involving the generation of active oxygen species (Vander Mijnsbrugge et al., 2000b).

Surprisingly, we found that, beside the known functions of PCBER, down-regulation of PCBER resulted in a lower lignin concentration and a higher biomass, especially a higher stem biomass. Moreover, downregulation of PCBER resulted in a higher concentration of soluble phenolics in the plant, which results in a higher resistance to plant pathogens, including, but not limited to herbivoric insects and fungal infection (Hwang and Lindroth, 1997).

One aspect of the invention is the use of PCBER to modulate plant biomass, compared to the plant biomass of a non-treated control. Preferably, said PCBER is originating from a plant selected from the group consisting of Betula pendula, Pinus taeda, Tsuga heterophylla, Thuja plicata, Forsythia x intermedia, Populus tricharpa, Solanum tuberosum, Nicotania tabacum, Zea mays, Arabidopsis thaliana, Pinus pinaster, Avicennia marina and Pyrus communes. More preferably, it is a PCBER enzyme from Populus balsamifera subsp. trichocarpa, even more preferably it is an enzyme comprising SEQ ID No. 2, even more preferably it is an enzyme essentially consisting of SEQ ID No. 2, most preferably it is an enzyme consisting of SEQ ID No. 2.

Preferably the plant in which the modulation of biomass is obtained is a tree. More preferably, said plant is a poplar tree.

In one preferred embodiment, said use is the repression of the activity of PCBER, resulting in an increase of plant biomass preferably an increase of plant stem biomass. Increase in biomass means every phenomenon that results in an increase of plant weight of a treated plant compared with an untreated control, and can be, as an unlimited example, an increase in stem thickness as well as an increase in height. Repression of the activity may be realized in any way known to the person skilled in the art, either, as a non limiting example, at protein level, e.g. by treatment of the plant with a PCBER inactivating compound, or by the expression of PCBER inactivating antibodies, at translation level, e.g. by expressing antisense RNA, at the transcription level, e.g. by inactivation of the promoter or by gene silencing, or at DNA level, e.g. by mutations in or by deletion of the gene. Preferably, said repression is realized by the expression of antisense RNA, more preferably, said repression is realized by RNA interference, even more preferably, said repression is realized by cosuppression. Preferably, said increase in plant biomass is accompanied by a lower lignin content and/or a higher concentration of soluble phenolics and/or a higher resistance to pathogens, preferably a higher resistance to herbivoric insects and/or to fungal infection, compared to the untreated control. Another preferred embodiment is the use of PCBER to modulate plant biomass, compared to the plant biomass of a non-treated control, whereby said modulation is obtained under elevated CO₂ concentration. Preferably, said use is the repression of the activity of PCBER, preferably by the expression of antisense RNA, even more preferably by RNAi, even more preferably by cosuppression resulting in an increase of plant biomass, preferably an increase of plant stem biomass. Preferably, said increase in plant biomass is accompanied by a lower lignin content and/or a higher concentration of soluble phenolics and/or a higher resistance to pathogens, preferably a higher resistance to herbivoric insects and/or to fungal infection, compared to the untreated control.

Another aspect of the invention is a method to modulate plant biomass, comprising the incorporation into the plant genome of the plant a recombinant nucleic acid encoding a phenylcoumaran benzylic ether reductase, or its complement, or a functional fragment thereof. fragment may be any fragment that is sufficient to obtain gene silencing, e.g. by cosuppression or which is effective as antisense RNA or as RNAi.

A preferred embodiment is the method, whereby the modulation of plant biomass is obtained by growth of the plant under elevated CO₂ concentration. Preferably, said modulation is an increase of plant biomass.

Still another aspect of the invention is a genetically modified plant, obtainable by the method according to the invention. Preferably, said genetically modified plant is expressing PCBER antisense RNA, more preferably, said genetically modified plant is expressing PCBER RNAi. Preferably, said genetically modified plant has an increased biomass, preferably an increased stem biomass.

In a preferred embodiment, said increased biomass is obtained under elevated CO₂ concentration. Preferably, said increase in plant biomass is accompanied by a lower lignin content and/or a higher concentration of soluble phenolics and/or a higher resistance to pathogens, preferably a higher resistance to herbivoric insects and/or to fungal infection, compared to the untreated control.

Preferably, said genetically modified plant is a tree, even more preferably, said genetically modified plant is a poplar tree.

Definitions

The following definitions are set forth to illustrate and define the meaning and scope of various terms used to describe the invention herein.

Phenylcoumaran benzylic ether reductase means any enzyme activity that can reduce the benzylic ether functionalities of both dehydrodiconiferyl alcohol and dihydrodehydrodiconiferyl alcohol, as measured and described by Gang et al. (1999); it does not exclude that the enzyme can be active on other substrates too, nor does it imply that the substrates mentioned are the preferential substrates. As a non-limiting example, it is chosen from a group of proteins from Betula pendula (Entrez protein accession number AAG22740, AAC05116), Pinus taeda (AAF64173), Tsuga heterophylla (AAF64185, AAF64184, AAF64182, AAF64181, AAF64180, AAF64179, AAF64178, AAF64177, AAF64176), Thuja plicata (AAF64183), Forsythia x intermedia (AAF64174, AAF64174), Populus balsamifera subsp. trichocarpa (CAA06709, CAA06708, CAA06707, CAA06706), Solanum tuberosum (P52578), Nicotania tabacum (P52579), Zea mays (P52580) or from a group of proteins encoded by a nucleic acid from Arabidopsis thaliana (genbank accession number NC_{—}003075, NM_{—}119619), Pinus pinaster (AL750375, AL750211), Avicennia marina (BM173321), Pyrus communis (AF071477) and Pinus taeda (AF081678). Preferably, it is a PCBER enzyme from Populus balsamifera subsp. trichocarpa, more preferably it is an enzyme comprising SEQ ID No. 2, even more preferably it is an enzyme essentially consisting of SEQ ID No. 2, most preferably it is an enzyme consisting of SEQ ID No. 2.

Repression of the activity of phenylcoumaran benzylic ether reductase as used here means that the activity of said enzyme is lower than that of a control plant, grown under the same conditions. The control depends upon the way of repression used; for a transgenic plant, the untransformed parental plant is used as control.

Elevated CO₂ concentration as used here, means any concentration that is significantly higher than the ambient concentration (385 ppm CO₂). Preferably, it is a concentration that is higher than 440 ppm CO₂. Even more preferably, it is higher than 700 ppm CO₂.

PCBER antisense RNA as used here means any RNA molecule that can hybridize with the PCBER mRNA molecule under physiological conditions, and is able to decrease the efficiency of translation of said mRNA molecule, as compared with a control where no such hybridisation can take place.

PCBER RNAi as used here means any RNA that can function in an RNA interference mechanism, amongst others described by Montgomery et a). (1998). Preferably, RNAi consists of short RNA fragments as disclosed in WO0244321.

### BRIEF DESCRIPTION OF THE FIGURES

FIG. 1

Schematic representation of the sense and antisense PCBER constructs. Only the T-DNA region is shown. The PCBERA coding sequence is shaded. Plasmids p70SSPCBER and p70SASPCBER contain the full length of the poplar PCBERA cDNA in sense or antisense orientation, flanked at the 5′end by a double CaMV 35S promoter (p70S) and at the 3′end by the terminator of the CaMV 35S gene (term CaMV). All plasmids contain the hpt gene as a selectable marker, under control of the nopaline synthase promoter (pNOS). LB, left border; RB, right border.

FIG. 2

Protein gel immunoblot of xylem harvested from two month old, greenhouse-grown poplars using ABPIL1 (polyclonal antibodies against poplar PCBER) in a dilution of 1/1000. As a control the blot was rehybridised with antibodies against CAD, an enzyme involved in the biosynthesis of lignin monomers. Anti-CAD (cinnamyl alcohol dehydrogenase) antibodies in a dilution of 1/1000. 20 μg of total protein was loaded on the gels. Wild type (WT) and transgenic lines are indicated above the lanes. The migration position of molecular weight markers is indicated. (SPIL: cosuppressed PCBER=SPCBER; ASPIL: antisense PCBER=ASPCBER)

FIG. 3

Stem biomass (dry mass per plant) of P. x canescens wild type (white columns) and the cosuppressed lines PCBER207 (black column, A) and PCBER201 (grey column, B) grown for 9-10 weeks in the greenhouse. Data indicate means (n=5-6 plants, ±SD).

FIG. 4

Nitrogen (% per dry mass; A) and soluble carbohydrates (sum of glucose, fructose, and sucrose, B) in the apical stem segment of P. x canescens wild type (white column) and PCBER down-regulated poplar line SPCBER201 (grey column). Data indicate means. (n=5-6 plants, ±SD).

FIG. 5

Mean PC1 value for each poplar line in each experiment. exp1, experiment 1; exp2, experiment 2.

FIG. 6.

Distribution of the coefficient values associated with PC1 for each of the 60 peaks.

FIG. 7

Lignin content in wood of wild type and transgenic poplars with suppressed PCBER. Lignin was determined with the Klason method in cell wall residuals of wood from five-month-old, greenhouse-grown wild type (white bars) and transgenic poplars down-regulated for PCBER (lines SPCBER207 (dark gray bars) and ASPCBER313 (light grey bars)) Three individual plants per line, grown in identical conditions, were analyzed. The data presented are means of two to four measurements per plant and are expressed as weight percentages of extractive-free cell wall residues (CWR). The standard errors are indicated.

FIG. 8

PCBER (A) and lignin (B) in basal stem parts of wild type (WT) poplar (P. x canescens (white columns) and in plants with suppressed PCBER (line SPCBER207: black columns; line SPCBER201: gray columns). Lignin was determined in the same tissues by the LTGA method (Materials and methods).

FIG. 9

Typical cross sections (40 μm) of lignifying (A) and differentiated xylem (B) taken from green, 1-to-2-week-old and from mature 7-to-8 week old stem segments of Populus x canescens, respectively. The sections were stained with phloroglucinol/HCL for lignin. Sections used for FTIR-microscopy are indicated by the frame. Cell wall-bound phenolics localized in cross sections (40 μm; C-F) of young poplar wood of the wildtype (C,E,F) and SPCBER201 (D). The trees were grown under elevated (F) or ambient CO₂-concentrations (C-E). Inset in C-D show details of typical vessels. The sections were stained with 0.1% (w/v) berberine-sulfate and photographed under UV-microscope. Magnifications: 10×25 (A), 10×40 (B), 10×20 (C-D), 10×10(E-F).

### EXAMPLES

Materials and Methods to the Examples

Plant Material, Transformation and Plasmid Constructions

Populus x canescens (P. tremula x P. alba, INRA clone 717 1 B-4) was chosen because this clone is easily propagated and transformed. In vitro plants were maintained on ½ MS medium at 22° C. with a photoperiod of 16 h light and 8 h darkness. Transformation of poplar was performed according to Leplé et al. (1992). For sense and antisense full-length constructs, an XbaI-KpnI fragment containing the full length PCBERA cDNA (Gang et al., 1999) was cut from the Bluescript II SK vector and cloned in pUC19 resulting in the plasmid pUCPCBER33. This plasmid was digested with EcoRI and cloned in pLBR19 in both directions, resulting in the plasmids called pLBRSPCBER (sense construct) and pLBRASPCBER (antisense construct). From these plasmids, XbaI-KpnI fragments containing the 70S promoter, the full length PCBERA sequence (in both directions) and the CaMV terminator sequence, were cut by a partial digest, and cloned in the binary vector pBIBHYG (Becker, 1990), giving rise to the plasmids p70SSPCBER (sense) and p70SASPCBER (antisense).

Growth Conditions and Plant Material for Metabolic Profiling

Wild type and transgenic poplars down-regulated for PCBER (SPCBER201, SPCBER207 and ASPCBER313) were propagated in vitro on MS medium (Murashige & Skoog, 1962) and plantlets were transferred to the greenhouse (21° C., 60% humidity, a 16/8 hour light/dark regime, 40-60 μmole m⁻² s⁻¹ photosynthetic photon flux). This experiment was done twice with 3 ramets for each line in the first experiment and 5 ramets in the second experiment.

HPLC Analysis of Soluble Phenolics

Xylem tissue was obtained by scraping a 20-cm long, debarked stem of six-month old poplars with a scalpel. After homogenisation in liquid nitrogen, extraction was done with 15 ml of methanol and samples were stored at −20° C. A 1-ml aliquot of the supernatants was freeze-dried for HPLC analysis. The subsequent liquid-liquid extraction, separation and chromatogram integration were performed as previously described (Meyermans et al., 2000). Quantification was based on the maximum absorbance value between 230 and 450 nm and expressed as % peak height, i.e. the height of the peak of interest relative to the sum of all peak heights in the chromatogram. Dehydrodiconiferyl alcohol (DDC) was identified within the chromatogram by spiking with a standard (kindly provided by A. Boudet).

Statistical Analyses of Soluble Phenolics

A principal component analysis (PCA) was done on all integrated peaks (60 peaks), followed by a Varimax rotation using the software SPSS10.0. The 7 major principal components (PC), explaining together approximately 80% of the variance of the initial data set, were retained and subjected to two-way ANOVA (α=0.001) followed by an LSD post hoc test (α=0.05) to reveal differences between wild type and each of the transgenic lines. Additionally, a two-way ANOVA (α=0.10) was applied to the % peak height of DOC.

Growth Conditions and Sampling for Growth Analysis

In initial experiments, rooted plantlets were potted in standard garden compost, transferred to greenhouse conditions (20° C.) with the same photoperiod as for the in vitro culture. For detailed characterization of wild type and transgenic poplars (line SPCBER201 and SPCBER207) rooted plantlets were preconditioned for two weeks in hydroponic culture in modified Long Ashton medium (pH: 5.5, Hewitt, 1966): 2.0 mM Ca (NO₃)₂, 200 μM KNO₃, 300 μM MgSO₄, 600 μM KH₂PO₄, 41.3 μM K₂HPO₄, 2 μM MnSO₄, 10 μM H₃BO₃, 7 μM Na₂MoO₄, 20 μM NaCl, 0.04 μM CoSO₄, 0.2 μM ZnSO₄, 0.2 μM CuSO₄, 10 μM EDTA, 5 μM FeCl₃. The medium was changed once a week.

Two week-old plants were potted in 3 liter garden compost type N (Fruhstorfer Erde, Industrie Erden Werk Archut, Lauterbach, Germany, line 11201 and controls) or in 3 liter sand and garden compost type N (1:1; v:v; line 11207 and controls) fertilized with 2 g of Osmocote Plus (Controlled Release Fertilizer, Scotts Deutschland GmbH, Nordhorn, Germany) per plant. The plants were watered as needed and once a weeks with fertilizer (Hakaphos Blau, Compos, Münster). After 6 weeks, poplars were potted into 6 l pots. The plants were kept under daylight and additional irradiation (HQL-MBF-U, 400 W, Osram, UK) yielding 200 to 300 μE m²s⁻¹ of photosynthetically active radiation at plant height 16 to 18 h and growth temperature of 24° C. (±1.76° C. during day and night).

During the experimental period: shoot length, leaf number and the diameter at the stem base were measured weekly. At harvest whole plant biomass and leaf area (WINDIA; Umweltanalytische Produkte, Ibbembüren, Germany) were also determined

Plants were harvested for biomass determination and biochemical analysis. Stems from 8-9 week-old plants were debarked and the young differentiating xylem was scraped off with a scalpel and kept frozen for Western blot analysis. Aliquots of debarked wood were frozen in liquid nitrogen and stored at −80° C. Further aliquots were dried for 120 h at 60° C. for biomass determination. All samples were taken in 4 to 6 replicates per experiment and line.

Plant Material, Culture Conditions, CO₂-Exposure and Sampling in Experiments Under Elevated CO₂.

Populus canescens, a hybrid of Populus tremula x alba wildtype plants (WT, INRA clone 717 1 B4) and the transgenic poplar line SPCBER201 (suppressed formation of PCBER protein, Vander Mijnsbrugge, 1998) were multiplied by micropropagation (Leplé et al., 1992). Rooted plantlets were preconditioned for two weeks in hydroponic culture in modified Long Ashton medium. The medium was changed once a week.

Two-week-old plants were potted in 3 liter garden compost type N (Fruhstorfer Erde, Industrie Erden Werk Archut, Lauterbach, Germany), fertilized with 2 g of Osmocote Plus (Controlled Release Fertilizer, Scotts Deutschland GmbH, Nordhom, Germany) per plant. The plants were transferred into greenhouse cabinets and exposed to ambient (385 ppm CO₂±55 ppm CO₂) or elevated (780 ppm CO₂±79 ppm CO₂) CO₂ concentrations, respectively. The CO₂-concentration was controlled for each treatment and adjusted every 35 min by injection of CO₂ (Widmann, Elchingen, Germany). In addition to daylight, the plants were irradiated from 6 am to 10 pm with Hg lamps (HQL-MBF-U, 400 W, Osram, Great Britain) yielding 200 to 300 μE m² s⁻¹ of photosynthetically active radiation at plant height to achieve 16 h day length. The temperature was maintained at 24.0±1.4° C. during day and night.

After nine weeks, stem sections of known age were harvested: wood with secondary growth (7-8 weeks, debarked) and young, elongating stem (1-2 weeks). Samples were taken in 6 replicates per treatment and line. Aliquots of the tissues were frozen in liquid nitrogen and stored at −80° C. for further analysis.

LTGA and Klason-Lignin Content Determination

The pellets obtained after extraction of the soluble phenolics were washed twice (10 min, 18000 g, 4° C.) with 2 ml n-hexane (Merck, Darmstadt, Germany), dried for two days at 70° C. and subsequently weighed. This fraction represented cell walls. The pellet was homogenized in four ml of 1 M NaOH. The suspension was de-aerated for 15 min by bubbling N₂ into the mixture (Messer, Griesheim, Germany). Subsequently, it was incubated for 60 min in an ultrasonic bath and centrifuged. The extraction was repeated. The pellets were washed twice with 2 ml distilled water, dried and used for lignin analysis. Lignins were determined by derivatization with thioglycolic acid (Blaschke et al. 2001; adapted after Bruce and West, 1989). Standard curves were produced with commercial lignin (alkaline spruce lignin; Sigma-Aldrich, Deisenhofen, Germany). Alternatively, Klason Lignin content of cell wall residuals was estimated according to the method of Effland (1977). For the latter, cell wall residuals are the dried residues obtained after successive extraction of the freeze-dried and ground wood with toluene:ethanol (2:1; v/v), ethanol and water.

Protein Extraction and Western Blot Analysis

Scraped tissues were extracted in 100 mM Tris-HCL pH 7.5, 2 mM EDTA and 20% glycerol. Protein concentrations of the extracts were measured after Bradford (1976) in supernatants after centrifugation (twice, 5 minutes). 15-20 μg protein per slot was loaded on SDS-gels. Immunodetection was performed according to the manufacturers instructions (Amersham, Aylesbury, U. K.) employing Tris-buffered saline Tween (TBS-T) instead of phosphate buffered saline Tween (PBS-T). Antibodies against poplar PCBER were used in a dilution of 1/1000 to detect the 37 kDa PCBER protein (Vander Mijnsbrugge et al., 2000a).

Carbohydrate and Nitrogen Analysis

For nitrogen analysis, dry plant material of young, expanding poplar shoots was milled. Aliquots of 0.6-1.0 mg were weighed into 5×9 mm tin cartouches (Hekatech, Wegberg, Germany) and analyzed with a CHNS-O-Elemental Analyzer (CHNS-O-EA1108-Elemental Analyzer, Carlo Erba Instruments, Rodano, Italy). Acetanilide (71.09% C, 10.36% N; Carlo Erba Instruments, Rodano, Italy).

For soluble carbohydrate analysis, frozen plant material of young, expanding shoots was ground in liquid nitrogen. The powder (30 mg) was boiled in 1 ml of water for 5 min in a heat block (Liebisch, Bielefeld, Germany). The extracts were centrifuged (4° C., 15000 g, 10 min; Mikro 24-48R, Hettich, Tuttlingen, Germany) and the supernatants were used for analysis of Sucrose, D-Glucose and D-Fructose employing an enzymatic test (Boehringer, Mannheim, Germany).

Lignin Determination of Plants Grown Under Elevated CO₂

Cell wall pellets obtained after extraction of cell wall-bound phenolics (NaOH-extraction) were washed twice with 2 ml distilled water, dried and used for lignin analysis. Lignins were determined by derivatisation with thioglycolic acid (adapted after Bruce and West 1989). Standard curves were produced with commercial lignin (alkaline spruce lignin, Sigma-Aldrich, Deisenhofen, Germany). To account for the difference in extinction coefficient for conifer (18.8) versus angiosperm lignins (14.4), the data were corrected by a factor of 1.32.

Light and Fluorescence Microscopy

40-μthick cross sections were cut with a sliding-microtome (Reichardt-Jung, Austria). The sections were stained with phloroglucinol/HCL for lignin localisation or with 0.1% (w/v) berberine-sulfate in water for localisation of phenolic compounds. The sections were photographed with a digital camera (Coolpix 990, Nikon, Tokyo) under a microscope (Axioplan microscope and UV filter UV-G365, both Zeiss, Germany).

FTIR-Microscopy

Stem sections (40 μm) were used for recording FT-IR spectra using a Fourier-Transform-Infrared microscope (Biorad, Digilab Division UMA). The spectrometer was equipped with a MCT- (mercury-cadmium-tellurid) detector cooled with liquid nitrogen. Sections were placed on a germanium plate on the stage of the FT-IR microscope. After focusing, the aperture was adjusted to frame only the desired portion for scanning. The transmission mode was used and 100 scans were accumulated to produce a spectrum over the 4000 to 700 cm⁻¹ wavenumber range at a resolution of 8 cm⁻¹. A background was scanned using the germanium plate free of any tissue and 500 scans were accumulated to produce the transmission spectrum in the 4000 to 700 cm⁻¹ range and displayed in absorbance mode. All measurements were repeated twice. After baseline and background correction with the Biorad Win-IR software the absorbance spectra were stored as Asc-II-files for further analysis.

The absorbance at wavenumber 1325-1330 cm⁻¹ due to syringyl ring breathing and the absorbance at wavenumber 1266-1275 cm⁻¹ due to guaiacyl ring breathing (Faix, 1992) were used to determine the ratio of S- and G-units of lignin. The band at 899 cm⁻¹, due to the anomeric C-O stretch in cellulose (Hergert, 1971) was chosen as the band to represent carbohydrate. The bands at 1740 cm⁻¹ (C=O stretch in unconjugated ketones, carbonyl and ester groups; Hergert, 1971; Faix, 1992) and at 1030-1085 cm⁻¹ (C-H, C-O deformations; Hergert 1971; Faix, 1992) were measured to ensure that changes in S- and G-bands are not caused by a shift to carbonyl groups.

Statistical Analysis

Statistical analysis was performed with the software STATGRAPHICs (STN, St. Louis, USA) using multivariate analysis of variance followed by a multiple range test (LSD) to evaluate significant effects. Data in the tables and figures indicate means of individual plants (n=5 to 6; ±SD).

### Example 1

### Construction of Sense and Antisense PCBER Vectors and Transformation of Poplar

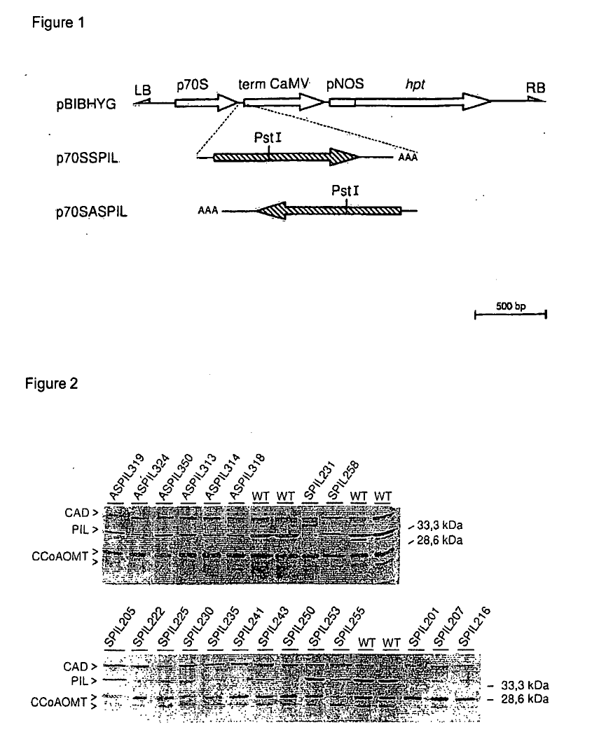

To address the biological function of PCBER, transgenic poplars were produced that expressed a full-length sense and a full-length antisense gene construct under the control of the CaMV 70S promoter. A schematic representation of the T-DNA constructs is shown in FIG. 1. Agrobacterium-mediated transformation of poplar yielded up to 50 transformants for each construct. Greenhouse-grown transgenic poplars were screened by protein gel immunoblot. A reduced amount of PCBER in the xylem was observed in 16 out of 48 plants analyzed for the sense construct (cosuppression) and in 7 out of 46 plants analyzed for the antisense construct.

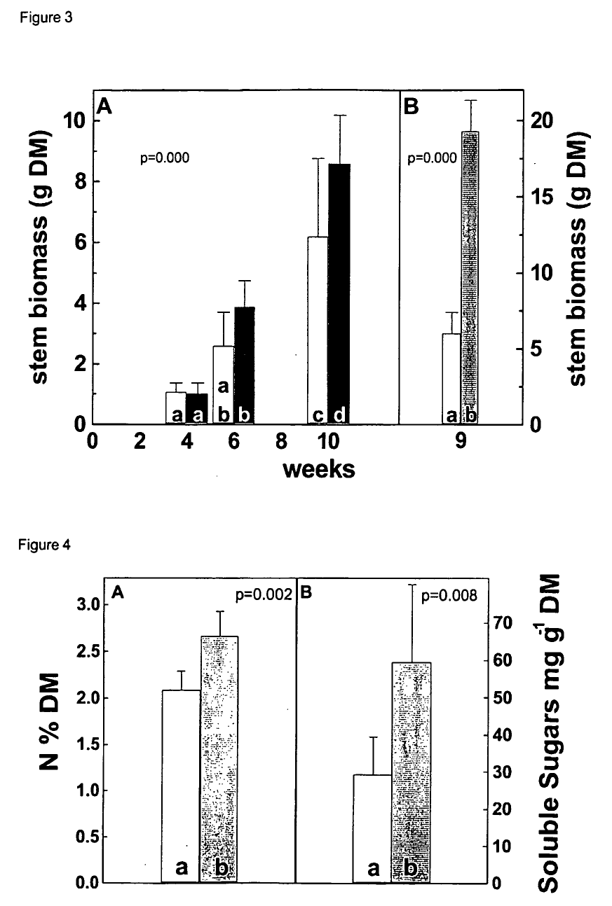

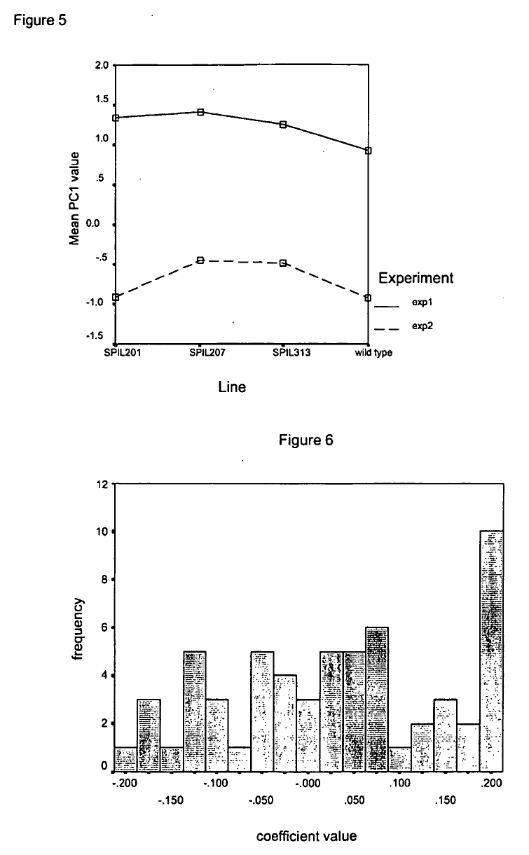

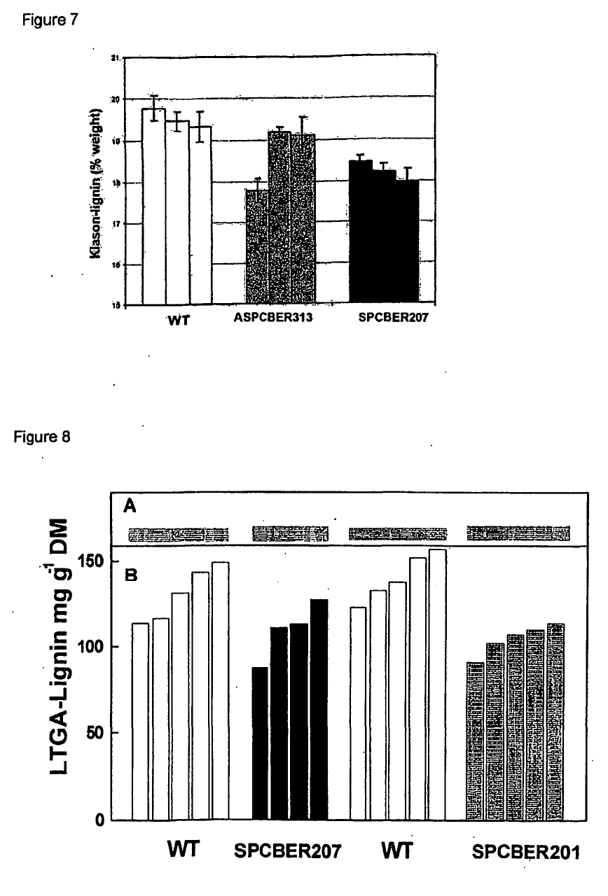

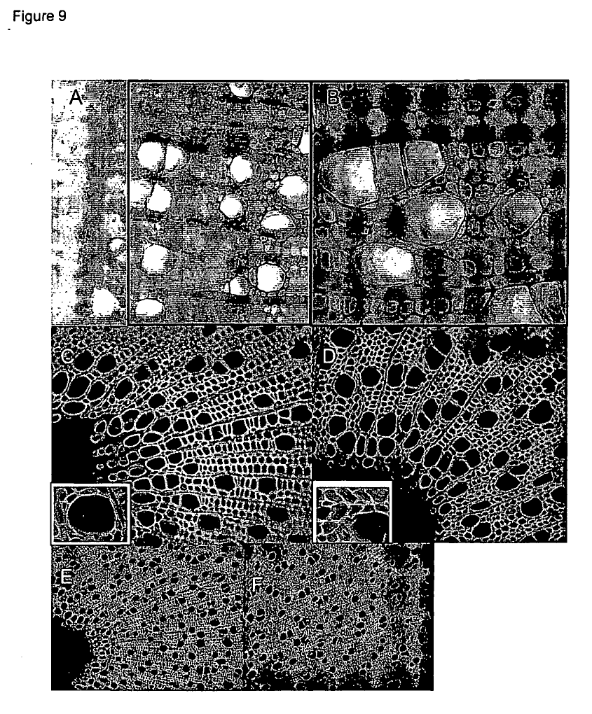

FIG. 2 shows that the cosuppressed lines had a stronger reduction in PCBER amount in the xylem than the antisense lines.

### Example 2

### Down-Regulation of PCBER Results in a Reduced Lignin Content in Wood

Because of the strong association of PCBER with lignifying cells (Vander Mijnsbrugge et al., 2000b), a possible function of PCBER in the lignification process was suggested. The lignin content in wood was measured by two different techniques. Analysis of Klason-Lignin of wood of five-month-old greenhouse-grown poplars showed that down-regulation of PCBER in the xylem through antisense (ASPCBER313) and cosuppression (SPCBER207) caused an up to 6% decrease in lignin content. Because the antisense line (ASPCBER313) showed a less pronounced reduction of PCBER in wood on immunoblot (FIG. 2) together with a smaller decrease in lignin content, the subsequent analyses were performed on cosuppression lines. Two lines, SPCBER201 and SPCBER207, with strong suppression of PCBER were chosen for further characterization of the lignin content. The wood of two-months old, greenhouse grown poplars was analyzed. The PCBER down-regulated poplars contained diminished LTGA-Lignin and Klason lignin concentrations compared to controls (FIG. 7, FIG. 8).

### Example 3

### Suppression of PCBER Results in Growth Stimulation and Reallocation of Internal Carbon and Nitrogen

Several reports indicate that modifications in the expression of genes involved in lignification affect plant morphology and growth (Hu et al., 1999; Maury et al., 1999; Pincon et al., 2001). The PCBER down-regulated poplars showed enhanced height as well as radial growth, which resulted in increased stem biomass production (FIG. 3 A, B; Table 1). We reasoned that growth acceleration of the apex would require an enhanced supply of carbohydrate and nitrogen resources. Apical stem segment of poplars with suppressed PCBER contained increased concentrations of both soluble carbohydrates (glucose+fructose+sucrose) and nitrogen (FIG. 4). This shows that the internal nutrient allocation pattern was shifted in plants with suppressed PCBER. The substrate or product of PCBER or related metabolites may function as phytohormones in poplar regulating the sink strength of the apex. Ploidy levels were equal in all three lines and the wild-type analyzed.

The growth stimulation in poplars with suppressed PCBER resulting in increased stem biomass is surprising. Less lignification and perhaps lower cell wall stiffening may enable stronger cell expansion. However, microscopic analysis of wood properties revealed no significant anatomical changes in PCBER suppressed poplar. The growth stimulation can be explained by the internal allocation of resources to growth instead of lignin formation. We found increased carbohydrate and nitrogen resources in apical stem segments of poplar with suppressed PCBER (Table 4). This indicates a stronger sink strength of the apex. We suspect that the allocation of increased nutrients including nitrogen to the apical stem tissue was not caused by general increases in internal nutrient resources in PCBER-suppressed poplars, because leaves, the major plant compartment for nitrogen, were relatively N-depleted in the transformed poplar. The tissue-specific re-allocation of internal resources by modulation of PCBER suggests the presence of factors controlling sink strength, thus enabling increased growth.

### Example 4

### Metabolite Profiling

To investigate whether the decreased consumption of phenylpropanoids for lignification had upstream effects on the accumulation of soluble phenolics, HPLC separations were performed of the methanol soluble phenolics present in xylem tissue of wild type and the transgenic poplar lines SPCBER201, SPCBER207 and ASPCBER313. For two independent experiments, the same 60 peaks were analyzed in both experiments. Considering all the resulting chromatograms, correlations found between the concentrations of these peaks are due to the use of % peak height for quantification and to co-variation between peaks as a result of the two experiments and of the down-regulation of PCBER. Applying Principal Component Analysis (PCA) reduced the initial data set of 60 peaks to 7 PC of which the first PC (PC 1) explained 35% of the variance (Table 2). To reveal differences in the values of these 7 PC between the poplar lines on one hand and between the two experiments on the other, a two-way ANOVA was executed for each PC. The ANOVA model was only significant in the case of PC1 (Table 3). Down-regulation of PCBER as well as the incorporation of two different experiments both attributed to this significance. The value of PC1 was increased in the transgenic poplar lines as compared to the wild types (FIG. 5).

The value of a PC is related to the concentration of each compound by means of the coefficient associated with that peak. These coefficients can be negative or positive, according to the effect of the concentration of the corresponding peak on the value of the PC, with a value of 0 representing no effect. Considering the magnitude of the coefficients associated with PC1, most of the 60 peaks contributed to this PC (FIG. 6). The UV/VIS spectra of the peaks having an extreme coefficient value, either positive or negative, ranged from simple phenolics to cinnamic acid derivatives and lignans. We therefore conclude that down-regulation of PCBER resulted in gross alterations of the phenolic metabolite spectrum instead of the accumulation of specific metabolites, such as dehydrodiconiferyl alcohol (DDC) and dihydrodehydrodiconiferyl alcohol (DDDC) which have been postulated as substrates of PCBER by means of in vitro studies (Davin et al., 1992). DDC affected the value of PC1 as well, as shown by its coefficient of 0.40. However, its contribution was of minor importance because at least 33% of the other peaks showed a more extreme coefficient (FIG. 6). An additional two-way ANOVA was applied to the % peak height of DDC. Even using a high significance threshold of 0.10, no differences in the mean % peak height value between any of the different poplar lines could be shown. Considering both experiments together, the mean % peak height value for DDC was lower in the transgenic poplars down-regulated for PCBER than in wild type poplars, oppositely to what would be expected for DDC being the substrate for PCBER.

### Example 5

### Wood Structure Under Elevated CO₂ in Wild Type and Transgenic Poplars

Cross sections of wild type poplar displaying typical examples for tissues used for biochemical and structural wood analysis of young elongating stem segments (1-to-2-weeks-old) and of about 7-to-8-week-old wood (differentiated xylem) are shown in FIG. 9 A and B. In elongating young tissues, lignification was mainly apparent in the corners of adjacent cells, especially around vessel (FIG. 9 A). The fibre cell walls just started to lignify (FIG. 9 A; vessel diameter 52 μm). In differentiated xylem, the walls of all cell types, namely, vessels, fibres and ray cells, displayed an intensive phloroglucinol staining indicating a high degree of lignification (FIG. 9 B). In wood of wild type poplar, growth under elevated CO₂ caused structural alterations in favour of parenchymatic and fibre cells and a lower abundance of vessels (−11%, FIG. 9 G as compared with FIG. 9 H).

Structural alterations due to elevated CO₂ were not found in transgenic poplars. Anatomical difference between the wild type and line SPCBER201 were not found (FIG. 9 F, C).

### Example 6

### Lignin Concentration and Composition Under Elevated CO₂ in Wild Type and Transgenic Poplars

Growth under elevated CO₂ concentrations resulted in a modest but significant diminution of cell wall lignification in young, elongating shoot tissue in wild type, but not in line SPCBER201. However, this line contained significantly lower lignin concentrations than the wild type (Table 5). In general, cell walls of 2-months-old wood contained about 2.6-fold higher lignin concentrations than cell walls isolated from young elongation shoots (Table 5).

To investigate whether the S/G-ratio of lignin was affected, when wild type and transgenic plants were grown under elevated CO₂, wood and young elongating stem tissues were analysed by FTIR spectroscopy. Because of the limited resolution of the FTIR microscope, analysis on single cell walls was not possible. To address age-related effects, xylem samples of defined developmental stages as indicated by the black frame in FIG. 9 A and B were chosen for analysis. FTIR spectra of wood showed the typical syringyl band at wave numbers of 1330-1325 cm⁻¹ and of the guaiacyl band at wave numbers of 1270-1275 cm⁻¹. Calculations of the S/G-ratio from the absorbance of syringyl- and guaiacyl-bands showed that poplar wood generally contained slightly higher concentrations of G- than of S-units since the S/G-ratio was usually lower than 1 (Table 5). Neither in wild type, not in SPCBER201, growth under elevated CO₂ caused significant changes in the S/G-ratio in differentiated xylem (Table 5).

The S/G-ratios showed, however, age-dependent shifts. For wild type, the S/G-ratio was decreased with increasing age of the xylem (Table 5) because of an increased portion of G-units, but the extent of this shift was moderate and corresponded to about 5% higher concentrations of G- than of S-units (Table 5).

Tables:

TABLE 1
Stem height (mm), stem area (mm²), leaf
initiation per day and total biomass (g FM) of wildtype (WT),
antisense CAD (T21) and cosuppressed PCBER (11201) poplars
grown under ambient (a) and elevated (e) CO₂ for
nine weeks. Data are means (n = 6, ±SD). LSD-Test was
used for MANOVA.

| | | Stem | Stem | Leaf initiation | Biomass |
|---|---|---|---|---|---|
| Line | [CO₂] | height (mm) | area (mm²) | rate (day⁻¹) | (g plant⁻¹) |
| WT | A | 795 ± 89a | 40 ± 8a | 0.88 ± 0.07a | 67 ± 14a |
| | E | 1055 ± 33b | 51 ± 4a | 0.99 ± 0.07b | 128 ± 13b |
| SPCBER201 | A | 1280 ± 62c | 81 ± 11b | 1.12 ± 0.08c | 185 ± 14c |
| | E | 1495 ± 114d | 132 ± 26c | 1.09 ± 0.04c | 274 ± 40d |

TABLE 2
The % of the variance of the initial data set explained
by the 7 retained principal components.

| Principal Component | % of Variance | Cumulative % |
|---|---|---|
| 1 | 35.282 | 35.282 |
| 2 | 19.213 | 54.495 |
| 3 | 9.207 | 63.701 |
| 4 | 7.718 | 71.419 |
| 5 | 5.294 | 76.713 |
| 6 | 3.487 | 80.200 |
| 7 | 2.531 | 82.731 |

TABLE 3
Two-way ANOVA table obtained for the first principal
component. Line effect and experiment effect indicates
how significant the values of this principal component
differ between the different poplar lines and between the
two experiments, respectively. The interaction of these
two effects is annotated as line × experiment.

| Source | Sum of Squares | df | Mean Square | F | Significance |
|---|---|---|---|---|---|
| Model | 23.124 | 7 | 3.303 | 65.717 | 1.62e−14 |
| Line effect | 0.801 | 3 | 0.267 | 5.314 | 5.68e−3 |
| Experiment effect | 22.100 | 1 | 22.100 | 439.642 | <1.00e−16 |
| Line × Experiment | 0.223 | 3 | 7.435e−2 | 1.479 | 0.24 |
| Error | 1.257 | 25 | 5.027e−2 | | |
| Total | 24.381 | 32 | | | |

TABLE 4
Concentrations of nitrogen (mg g⁻¹ DM) in
elongating shoot of P. x canescens
wild type (WT) and cosuppressed PCBER-plants (line SPCBER201
and line SPCBER207). Data indicate means (±SD; n = 5-6).

| | | statistics | | | statistics |
|---|---|---|---|---|---|
| WT | SPCBER201 | p₋ᵥₐₗᵤₑ | WT | SPCBER207 | p₋ᵥₐₗᵤₑ |
| 2.09 ± 0.20 | 2.67 ± 0.26 | 0.002 | 2.87 ± 0.11 | 3.05 ± 0.24 | 0.165 |

TABLE 5
LTGA-Lignin (lignothioglycolic acid-lignin), ratio of syringyl
(S) to guaiacyl (G)-units, ratio of S-lignin to carbohydrates
and ratio of C—H, C—O deformations to carbonyl-groups
in lignifying (1-2 weeks old young shoot) and differentiated
xylem (7-8-week-old wood) of Populus x
canescens wildtype plants and the transgenic
lines SPCBER201 (cosuppressed PCBER) grown under ambient
(A) or elevated (E) CO₂-concentrations. Data
were calculated on the basis of typical syringyl ring breathing
(1325-1330 cm⁻¹), guaiacyl ring breathing
(1270-1275 cm⁻¹), carbohydrate (899
cm⁻¹), carbonyl (1740 cm⁻¹)
and C—H, C—O deformation (1050 cm⁻¹) bands
identified in Fourier transform infrared spectras. Data are
means (n = 4 to 6, ±SD,). Different letters indicate
significant effects at P < 0.05 of each tissue. LSD-Test was
used for Manova including data of young shoot and mature wood.

| | | | LTGA-Lignin | | | |
|---|---|---|---|---|---|---|
| Tissue | Line | [CO₂] | (mg g⁻¹ CW) | S/G ratio | S/carbohydrates | 1050/1740 |
| Young | WT | A | 98.75 ± 24.77cd | 0.918 ± 0.072ab | 1.28 ± 0.20a | 1.70 ± 0.50ab |
| shoot | | E | 84.54 ± 6.07abc | 0.958 ± 0.019b | 1.51 ± 0.55ab | 1.41 ± 0.44a |
| | SPC | A | 75.31 ± 7.51a | 0.912 ± 0.048ab | 1.74 ± 0.40abc | 1.94 ± 0.36b |
| | BER | E | 71.43 ± 13.71a | 0.922 ± 0.017ab | 2.22 ± 0.62c | 1.36 ± 0.31a |
| | 201 | | | | | |
| Mature | WT | A | 232.70 ± 20.30c | 0.979 ± 0.025c | 2.55 ± 1.18b | 1.77 ± 0.30a |
| wood | | E | 218.46 ± 7.62c | 0.923 ± 0.012bc | 1.53 ± 0.26a | 2.12 ± 0.43a |
| | SPC | A | 152.63 ± 8.83a | 0.911 ± 0.050b | 1.29 ± 0.33a | 1.93 ± 0.45a |
| | BER | E | 157.0 ± 11.45a | 0.919 ± 0.042bc | 1.77 ± 1.01ab | 2.07 ± 0.65a |
| | 201 | | | | | |

| | | | | |
|---|---|---|---|---|
| p_{Age} | 0.000 | 0.008 | 0.470 | 0.000 |
| p_{CO2} | 0.000 | 0.677 | 0.925 | 0.325 |
| p_{Line} | 0.000 | 0.000 | 0.792 | 0.868 |

### REFERENCES

Adlercreutz, H. and Mazur, W. (1997): Phyto-oestrogens and western diseases. Annuals of Medicine 29, 95-120
Ayres, D. S. and Loike, J. D. (ed) (1990): Lignans: chemical, biological and clinical properties (Chemistry and Pharmacology of natural Products). Cambridge University Press, Cambridge
Becker, D. (1990): Binary vectors which allow the exchange of plant selectable markers and reporter genes. Nucleic Acids Research 18, 203
Binns, A. N., Chen, R. H., Wood, H. N., Lynn, D. G. (1987): Cell division promoting activity of naturally occurring dehydrodiconiferyl glucosides: do cell wall components control cell division? Proceedings of National Academy of Sciences USA 84, 980-984
Blaschke, L., Schulte, M., Raschi, A., Slee, N., Rennenberg, H. and Polle, A. (2001): Photosynthesis, soluble and structural carbon compounds in two mediterranean oak species (Quercus pubescens and Quercus ilex) after lifetime growth at naturally enhanced CO₂ concentrations. Plant Biology 3, 288-297
Bradford, M. M. (1976): A rapid and sensitive method for the quantitation of microgram quantities of protein utilizing the principle of protein-dye binding. Annals of Biochemistry 72, 248-254
Bruce R. J., West C. A. (1989) Elicitation of lignin biosynthesis and isoperoxidase activity by pectic fragments in suspension cultures of castor bean. Plant Physiol 31: 241-247
Davin, L. B., Lewis, N. G., and Umezawa, T. (1992): Phenylpropanoid metabolism: biosynthesis of monolignols, lignans and neolignans, lignins and suberins. In: Phenolic Metabolism in Plants, (Recent Advances in Phytochemistry, Vol. 26), H. A. Stafford, and R. K. Ibrahim (Eds.). New York, Plenum Press, pp. 325-375
Effland, M. J. (1977): Modified procedure to determine acid-insoluble lignin in wood and pulp. Tappi 60,143-144.
Faix, O., Bremenr, J., Meier, D., Fortman, I., Scheijen, M. A., and Boon, J. J. (1992). Characterisation of tobacco lignin by analytical pyrolysis and fourier Transform Infrared Spectroscopy J. Anal. Appl. Pyrolysis 22, 239-259.
Gang, D. R., Kasahara, H., Xia, Z. O., Vander Mijnsbrugge, K., Bauw, G., Boerjan, W., Van Montagu, M., Davin, L. B., Lewis, N. G. (1999): Evolution of Plant Defense Mechanisms. Relationships of phenylcoumaran benzylic ether reductase to pinoresinol-lariciresinol and isoflavone reductases. Journal of Biological Chemistry, 274, 7516-7527
Gaspar, T., Kevers, C., Penel, C., Greppin, H., Reid, D. M., Thorpe, T. A. (1996): Plant hormones and plant growth regulators in plant tissue culture. In Vitro Cellular and Developmental Biology Plant 32, 272-289
Hewitt E. J. (1966): Sand and water culture methods used in the study of plant nutrition, 2nd revised edition, Commonwealth Bureau of Horticulture and Plantation Crops, East Malling, Technical communication No. 22, pp. 431-432, Commonwealth Agriculture Bureau, Farnham Royal, U.K.
Higushi, T. (1997): Biochemistry and Molecular Biology of Wood. Springer Series in Wood Science, 1997
Hillis, W. E. (ed) (1987): Heartwood and tree exudates (Spronger Series in Wood Science). Springer, Berlin
Hu, W. J., Harding, S. A., Lung, J., Popko, J. L., Ralph, J., Stokke, D. D., Tsai, C. J., Chiang, V. L. (1999): Repression of lignin biosynthesis promotes cellulose accumulation and growth in transgenic trees. Nature Biotechnology 17, 808-812
Hwang, S. Y. and Lindroth, R. L. (1997) Clonar variation in follar chemistry of aspen: effects on gyspy moths and forest ten caterpillars. Oecologia, 111, 89-108.
Kwon, M., Davin, L. B., Lewis, N. G. (2001): In situ hybridization and immunolocalization of lignan reductases in woody tissues: implications for heartwood formation and other forms of vascular tissue preservation. Phytochemistry 57, 899-914
Leplé, J. C., Brasileiro, A. C. M., Michel, M. F., Delmotte, F., Jouanin, L. (1992): Transgenic poplars: expression of chimeric genes using four different constructs. Plant Cell Report 11: 137-141
Maury, S., Geoffroy, P. and Legrand, M. (1999): Tobacco O-methyltransferase involved in phenylpropanoid metabolism. The different caffeoyl-coenzyme A/5-hydroxyferuloyl-coenzyme A/3/5-O-methyltransferase and caffeic acid/5-hydroxyferulic aicd 3/5-O-methyltransferase classes have distinct substrate specificities and expression patterns. Plant Physiology 121, 215-223
Meyermans, H., Morreel, K., Lapierre, C., Pollet, B., De Bruyn, A., Busson, R., Herdewijn, P., Devreese, B., Van Beeumen, J., Marita, J., Ralph, J., Chen, C., Burggraeve, B., Van Montagu, M., Messens, E., and Boerjan, W. (2000): Modification in lignin and accumulation of phenolic glucosides in poplar xylem upon down-regulation of caffeol-Co A-O-methyltransferase, an enzyme involved in lignin biosynthesis. J. Biol. Chem. 275, 36899-36909
Montgomery, M. K., Xu, S. Q. and Fire, A. (1998). RNA as a target of double-stranded RNA-mediated genetic interference in Caenorhabditis elegans. Proc. Natl. Acad. Sci. USA 95, 15502-15507.
Murashige, T. and Skoog, F. (1962). A revised medium for rapid growth and bio assays with tobacco tissue cultures. Physiol. Plant. 15, 473-497.
Orr, J. D. and Lynn, D. G. (1992): Biosynthesis of dehydrodiconiferyl alcohol glucosides: implications for the control of tobacco cell growth. Plant Physiology 98, 343-352
Osbourne, A. E. (1999): Antimicrobial phytoprotectans and fungal pathogens: a commentary. Fungal-Genetics and Biology 26, 163-168
Pincon, G., Chabannes, M., Lapierre, C., Pollet, B., Ruel, K., Joseleau, J. P., Boudet, A. and Legrand, M. (2001): Simultaneous down-regulation of caffeic/5-hydroxy ferulic acid-O-methyltransferase I and cinnamoyl-coenzyme A reductase in the progeny from a cross between tobacco lines homozygous for each transgene. Consequences for plant development and lignin synthesis. Plant Physiology 126, 145-155
Sterky, F., Regan, S., Karlsson, J., Hertzberg, M., Rohde, A., Holmberg, A., Amini, B., Bhalerao, R., Larsson, M., Villarroel, R., Van Montagu, M., Sandberg, G., Olsson, O., Teeri, T. T., Boerjan, W., Gustafsson, P., Uhlen, M., Sundberg, B. and Lundeberg, J. (1998): Gene discovery in the wood-forming tissues of poplar: analysis of 5, 692 expressed sequence tags. Proceedings of the National Academy of Science USA, 95, 13330-13335.
Tamagogne, L., Merida, A., Stacey, N., Plaskitt, K., Chang, C. F., Lynn, D., Dow J. M., Roberts, K. and Martin, C. (1998): Inhibition of phenolic acid metabolism results in precocious cell death and altered cell morphology in leaves of transgenic tobacco plants. The Plant Cell 10, 1801-1816
Vander Mijnsbrugge, K. (1998): Molecular biology approaches to study xylogenesis in poplar. Phd thesis, Faculteit Wetenschappen, Vakgroep Moleculaire Genetica, Laboratorium voor Genetica, Universiteit Gent, Belgien
Vander Mijnsbrugge, K., Meyermans, H., Van Montagu, M., Bauw, G., Boerjan, W. (2000a): Wood formation in poplar: identification, characterization, and seasonal variation of xylem proteins. Planta 210, 589-598
Vander Mijnsbrugge, K., Beeckman, H., De Rycke, R., Van Montagu, M., Engler, G., Boerjan, W. (2000b): Phenylcoumaran benzylic ether reductase, a prominent poplar xylem protein, is strongly associated with phenylpropanoid biosynthesis in lignifying cells. Planta 211, 502-509
Wards, R. S. (1997): Lignans, neolignans and related compounds. Nat Prod Report 14, 43-74

1. The use of phenylcoumaran benzylic ether reductase to modulate plant biomass.

2. The use according to claim 1, whereby said phenylcoumaran benzylic ether reductase comprises SEQ ID NO:2.

3. The use according to claim 1, whereby said use is a repression of the activity of phenylcoumaran benzylic ether reductase.

4. The use according to claim 3, whereby said repression of the activity is obtained by cosuppression RNAi.

5. The use according to claim 3, whereby said repression of the activity is obtained by antisense RNA.

6. The use according to claim 1, whereby said modulation is an increase of plant biomass.

7. The use according to claim 6, whereby said increase of plant biomass is an increase in plant stem biomass.

8. The use according to claim 6, whereby said increase of biomass is combined with a lower lignin content.

9. The use according to claim 6, whereby said increase is combined with a higher resistance to plant pathogens.

10. The use according to claim 1, whereby said plant is a tree.

11. The use according to claim 10, whereby said tree is a poplar tree.

12. The use according to claim 1, whereby said plant is grown under elevated CO₂ concentration.

13. A method to modulate plant biomass, comprising the incorporation into the plant genome of a recombinant nucleic acid encoding a phenylcoumaran benzylic ether reductase, or its complement, or a functional fragment thereof.

14. The method of claim 13, whereby said modulation is obtained under elevated CO₂ concentration.

15. A genetically modified plant, obtainable by the method of claim 13.

16. A genetically modified plant according to claim 15, expressing phenylcoumaran benzylic ether reductase antisense RNA.

17. A genetically modified plant according to claim 15, expressing phenylcoumaran benzylic ether reductase RNAi.

18. A genetically modified plant according to claim 15, whereby said plant has an increased biomass.

19. A genetically modified plant according to claim 18, whereby said increased biomass is increased stem biomass.

20. A genetically modified plant whereby according to claim 15, whereby said increased biomass is obtained under elevated CO₂ concentration.

21. A genetically modified plant, according to claim 15, whereby said plant has a lowered lignin content.

22. A genetically modified plant, according to claim 15, whereby said plant has an increased resistance to plant pathogens.

23. A genetically modified plant according to claim 15, whereby said plant is a tree.

24. A genetically modified plant according to claim 23, whereby said plant is a poplar tree.
